(19) United States
(12) Patent Application Publication
(10) Pub. No.: US 2025/0221973 A1
(43) Pub. Date: Jul. 10, 2025

(54) METHOD AND SYSTEM FOR DELIVERING AN ACTIVE INGREDIENT BY INHALATION

(71) Applicant: BLUE BLOOD BIOTECH CORPORATION, TAIPEI CITY (TW)

(72) Inventors: Chien-Chen Lin, New Taipei City (TW); Jiun-Yan Ding, Taoyuan City (TW)

(73) Assignee: BLUE BLOOD BIOTECH CORPORATION, TAIPEI CITY (TW)

(21) Appl. No.: 18/404,851

(22) Filed: Jan. 4, 2024

(51) Int. Cl. A61K 31/4412; A61K 9/00; A61K 9/107; A61K 9/12; A61K 47/14; A61M 15/00

(52) U.S. Cl. CPC A61K 31/4412; A61K 9/0043; A61K 9/107; A61K 9/12; A61K 47/14; A61M 15/0001

(57) ABSTRACT

A method for delivering an active ingredient by inhalation is provided. The method includes atomizing a mixture of the active ingredient, an aqueous solution, and a lipid carrier into an aerosol; and administering an effective amount of the active ingredient to a subject in need thereof by inhalation of the aerosol into a respiratory system of the subject. Also provided is delivering system for delivering an active ingredient to a respiratory system of a subject in need thereof by inhalation, including the mixture of the active ingredient, an aqueous solution, and a lipid carrier; and a pharmaceutically acceptable atomizer, nebulizer, or inhaler for atomizing the mixture into an aerosol.

### BACKGROUND OF THE INVENTION

### 1. Field of the Invention

The present disclosure relates to drug delivery, particularly to inhaled drug delivery.

### 2. Description of the Prior Art

Particle pollution, which involves particles of solids or liquids in the air, has become a serious problem in the industrialized societies. With the recent deterioration of air quality, substances in the air, such as the particulate matter with diameter less than or equal to 2.5 micrometers (PM 2.5), pose serious health hazard and cause respiratory diseases, e.g., asthma, chronic obstructive pulmonary disease (COPD), and lung cancer. In addition, the global COVID-19 pandemic caused by coronavirus results in pneumonia, and thus treatment strategy of lung diseases, especially the sequelae caused by pulmonary fibrosis, has attracted attention. Developing agent for treating lung injury and related diseases thereof has now become the focus of the pharmaceutical industry, among those, inhalant medication for the treatment of lung diseases has the characteristics of quick acting on the lungs by delivering through the respiratory system.

Therefore, in order to effectively improve the concentration of medication in serum or lung of a subject, there exists an unmet need in effectively delivering the medication by inhalation.

### SUMMARY OF THE INVENTION

In view of the foregoing, the present disclosure provides a method for delivering an active ingredient by inhalation, comprising atomizing a mixture of the active ingredient, an aqueous solution, and a lipid carrier into an aerosol, and administering an effective amount of the active ingredient to a subject in need thereof by inhalation of the aerosol into a respiratory system of the subject.

Also provided herein is a delivering system for delivering an active ingredient to a respiratory system of a subject in need thereof by inhalation, comprising a mixture of the active ingredient, an aqueous solution, and a lipid carrier; and a pharmaceutically acceptable atomizer, nebulizer, or inhaler for atomizing the mixture into an aerosol.

In the present disclosure, the method and system provided herein deliver the active ingredient of the drug into a respiratory system of a subject by inhalation, and effectively increase the drug concentration in serum or lung of a subject. In the present disclosure, the lipid carrier effectively brings about a better effect on delivering active ingredient, enhancing drug concentration in the subject as compared with those carried by other carriers. For instance, during the delivering, the active ingredients carried by those other than lipid may cause low efficacy due to their physical characteristics during inhalation. By contrast, the lipid carrier used in the present disclosure can achieve drug delivering by inhalation due to the lipid emulsion through emulsification of the active ingredients. In some embodiment, the lipid emulsion shows more superior effect on promoting drug delivery, enhancing drug concentration in the subject as compared with other emulsions.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present disclosure can be more fully understood by reading the following descriptions of the embodiments, with reference made to the accompanying drawings.

FIG. 1A is a photograph showing experimental groups of the Lipoplus® (LP)-containing mixture before atomizing characteristic evaluation.

FIG. 1B is a photograph showing the atomizing characteristic of the aerosol of the present disclosure. (a) PBS, (b) LP/PBS=1/2.5, (c) LP/PBS=1/5, and (d) LP/PBS=1/10.

FIG. 2A and FIG. 2B are photographs showing position where the cotton were placed. The number in FIG. 2A indicated the 3 locations where the cottons were deposited. As shown in FIG. 2B, the cottons were set on the bottom of the cylinder.

FIG. 3 is a bar chart showing the PFD concentration at different dilution ratio for cotton.

FIG. 4A is a photograph showing the experimental design for the mice. The position of the cotton (shown in FIG. 2A) were replaced by mice. After administering, the mice were sacrificed, and blood and lung thereof were preserved for further analysis.

FIG. 4B and FIG. 4C are graphs showing formulated concentration at different dilution rations in the lung and blood of the mice.

FIG. 4D and FIG. 4E are graphs showing formulated concentration vs. actual concentration of the Pirfenidone (PFD) in the lung and blood of the mice at different time points.

### DETAILED DESCRIPTION

The technical solutions illustrated in the examples of the present disclosure will now be described more clearly and completely, and it will be apparent that the described examples are merely part of the examples of the present disclosure and are not intended to be exhaustive. The present disclosure can also be implemented or applied as described in different examples. All other examples obtained without creative work by those skilled in the art are within the scope of the present disclosure.

It is further noted that, as used in this disclosure, the singular forms “a,” “an,” and “the” include plural referents unless expressly and unequivocally limited to one referent. The term “or” is used interchangeably with the term “and/or” unless the context clearly indicates otherwise.

As used herein, the phrase “at least one,” in reference to a list of one or more elements, should be understood to mean at least one element selected from any one or more of the elements in the list of elements, but not necessarily including at least one of each element listed within the list of elements and not excluding any combinations of elements in the list of elements. This definition also allows that elements may optionally be present other than the elements identified within the list of elements to which the phrase “at least one” refers, whether related or unrelated to those elements identified. Thus, as a non-limiting example, “at least one of A and B” (or, equivalently, “at least one of A or B,” or, equivalently, “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements).

As used herein, the term “comprising,” “comprises” “include,” “including,” “have,” “having,” “contain,” “containing,” and any other variations thereof are intended to cover a non-exclusive inclusion. For example, when describing an object “comprises” a limitation, unless otherwise specified, it may additionally include other elements, components, structures, regions, parts, devices, systems, steps, or connections, etc., and should not exclude other limitations.

The numeral ranges used herein are inclusive and combinable, and any numeral value that falls within the numeral scope herein could be taken as a maximum or minimum value to derive the sub-ranges therefrom. For example, it should be understood that the numeral range “10% to 20%” comprises any sub-ranges between the minimum value of 10% to the maximum value of 20%, such as the sub-ranges from 10% to 12%, from 18% to 20%, and from 11.5% to 19.5%. In addition, a plurality of numeral values used herein can be optionally selected as maximum and minimum values to derive numerical ranges. For instance, the numerical ranges of 10% to 20%, 10% to 15%, and 15% to 20% can be derived from the numeral values of 10%, 15%, and 20%.

The term “about” as used herein when referring to the numerical value is meant to encompass variations of ±20%, ±10%, ±5%, ±1%, ±0.5%, or ±0.1% from the numerical value. Such variations in the numerical value may occur by, e.g., the experimental error, the typical error in measuring or handling procedure for making compounds, compositions, concentrates, or formulations, the differences in the source, manufacture, or purity of starting materials or ingredients used in the present disclosure, or like considerations.

As used herein, the term “preventing” or “prevention” refers to preventive or avoidance measures for a disease or symptoms or conditions of a disease, which include, but are not limited to, applying or administering one or more active agents to a subject who has not yet been diagnosed as a patient suffering from the disease or the symptoms or conditions of the disease but may be susceptible or prone to the disease. The preventive measures of the present disclosure are provided to avoid, prevent, or postpone the occurrence of the disease or the symptoms or conditions of the disease.

As used herein, the term “treating” or “treatment” refers to obtaining a desired pharmacologic and/or physiologic effect, e.g., inhibition of viral entry and/or replication in a host. The effect may be prophylactic in terms of completely or partially preventing a disease or symptoms or conditions thereof or may be therapeutic in terms of completely or partially curing, alleviating, relieving, remedying, or ameliorating a disease or an adverse effect attributable to the disease or symptoms or conditions thereof.

As used herein, the terms “patient” and “subject” are used interchangeably. The term “subject” means a human or animal. Examples of the subject include, but are not limited to, human, monkey, mice, rat, woodchuck, ferret, rabbit, hamster, cow, horse, pig, deer, dog, cat, fox, wolf, chicken, emu, and ostrich. In at least one embodiment of the present disclosure, the subject is a mammal, e.g., a primate such as a human.

As used herein, the phrase “an effective amount” refers to the amount of an active agent that is required to confer a desired preventive or therapeutic effect on a subject in need thereof (e.g., reducing the amount of viruses in a host). Effective doses may vary, as recognized by those skilled in the art, depending on routes of administration, excipient usage, the possibility of co-usage with other therapeutic treatment, and the condition to be treated.

As used herein, the term “administering” or “administration” refers to the placement of an active ingredient into a subject by a method or route which results in at least partial localization of the active ingredient at a desired site to produce the desired effect. For example, the active ingredient of the present disclosure is administered to the subject by inhalation.

The present disclosure is directed to a method for delivering the active ingredient of the drug by inhalation.

In at least one embodiment of the present disclosure, the aqueous solution comprises a buffer. In at least one embodiment of the present disclosure, the lipid carrier comprises a lipid emulsion. In some embodiments, the lipid emulsion comprises at least one lipid selected from the group consisting of Smoflipid®, Lipofundin®, Lipovenoes®, Lipovenos®, Intralipid®, Lipoplus®, and any combination thereof. In some embodiments, the lipid emulsion comprises more than one lipid selected from the group consisting of Smoflipid®, Lipofundin®, Lipovenoes®, Lipovenos®, Intralipid®, Lipoplus®, and any combination thereof.

In some embodiments, the lipid has a concentration of about 10% to about 20% based on a total volume of the lipid carrier, e.g., the lipid has a concentration of about 10% or about 20% based on the total volume of the lipid carrier. In some embodiments, the lipid emulsion comprises Lipoplus®.

In at least one embodiment of the present disclosure, the lipid carrier comprises an ingredient selected from the group consisting of soybean oil, medium-chain fatty acid, olive oil, fish oil, ω-3 oil, glycerol, lecithin, α-tocopherol, sodium oleate, palmitate, and any combination thereof. In some embodiments, the soybean oil has a concentration of about 0% to about 20% based on the total volume of the lipid carrier. In some embodiments, the medium-chain fatty acid has a concentration of about 0% to about 20% based on the total volume of the lipid carrier. In some embodiments, the olive oil has a concentration of about 0% to about 10% based on the total volume of the lipid carrier. In some embodiments, the fish oil has a concentration of about 0% to about 5% based on the total volume of the lipid carrier. In some embodiments, the ω-3 oil has a concentration of about 0% to about 3% based on the total volume of the lipid carrier. In some embodiments, the glycerol has a concentration of about 0% to about 5% based on the total volume of the lipid carrier. In some embodiments, the lecithin has a concentration of about 0% to about 2% based on the total volume of the lipid carrier. In some embodiments, the α-tocopherol has a concentration of about 0.01% to about 0.15% based on the total volume of the lipid carrier. In some embodiments, the sodium oleate has a concentration of about 0.01% to about 0.5% based on the total volume of the lipid carrier. In some embodiments, the palmitate has a concentration of about 0.01% to about 0.5% based on the total volume of the lipid carrier.

In at least one embodiment of the present disclosure, the respiratory system comprises a respiratory tract or a lung. In some embodiments, the respiratory system comprises oral cavity, nasal cavity, pharynx, larynx, trachea, carina, primary bronchi, secondary bronchus, tertiary bronchi, bronchioles, alveoli, or any combination thereof.

In at least one embodiment of the present disclosure, the buffer is a solution having a pH value of about 6.0 to about 8.0. In at least one embodiment of the present disclosure, the buffer is a saline solution comprising at least one pharmaceutically acceptable salt of borate, citrate, bicarbonate, carbonate, glutamate, lactate, malate, phosphate, acetate, alginate, aspartate, sulfonate, fumarate, lactobionate, laurate, maleate, or palmitate. In at least one embodiment of the present disclosure, the buffer is an organic compound solution comprises at least one pharmaceutically acceptable amine of glycine, methionine, monoethanolamine, diethanolamine, 2-Amino-2-hydroxymethyl-propane-1,3-diol, triethylamine, procaine, dibenzylamine, N-benzyl-β-phenethylamine, 1-ephenamine, N,N′-dibenzylethylene-diamine, dehydroabietylamine, N-ethylpiperidine, benzylamine, or dicyclohexylamine.

In at least one embodiment of the present disclosure, the active ingredient is a predetermined class of a small molecule with molecular weight≤1000 daltons and may regulate a biological process. In some embodiments, the small molecule comprises a natural ingredient, artificial ingredient, or autologous ingredient. In at least one embodiment of the present disclosure, the active ingredient is a predetermined class of a large molecule with molecular weight >1 kilodaltons (kDa) (e.g., 100 kDa, 200 kDa, 400 kDa, 400 kDa, 1 500 kDa, 600 kDa, 700 kDa, 800 kDa, 900 kDa, and 1000 kDa) and may regulate a biological process.

In some embodiments, the large molecule comprises a natural ingredient, an artificial ingredient, or an autologous ingredient. In at least one embodiment of the present disclosure, the active ingredient is at least one selected from the group consisting of Pirfenidone (PFD), Nintedanib, mRNA, steroid, anticoagulants, immunosuppressive drugs/agents, endothelin receptor antagonist, phosphodiesterase inhibitor, colchicine, chemotherapeutic agents, peptide, and protein. In some embodiments, the active ingredient is Pirfenidone (PFD) or Nintedanib.

In at least one embodiment of the present disclosure, the lipid carrier comprises a lipid emulsion, the buffer is a solution having a pH value of about 6.0 to about 8.0, and the lipid carrier present in the mixture in an amount ranging from volume percentage about 0.5% to about 40%.

In at least one embodiment of the present disclosure, the aerosol is atomized by a pharmaceutically acceptable atomizer, nebulizer, or inhaler.

In at least one embodiment of the present disclosure, the particle size of the aerosol in the presence of the lipid carrier is less than 5 μm which can be delivered to alveoli. In at least one embodiment, the aerosol in the presence of the lipid carrier brings about a better effect on delivering active ingredient as compared with that in the presence of other carriers such as polyethylene glycol 1000 (PEG 1000), polyethylene glycol 400 (PEG 400), povidone, polysorbate 80, butylated hydroxytoluene (BHT), and butylated hydroxyanisole (BHA).

In some embodiment, the aerosol in the presence of the lipid carrier shows more superior effect on delivering an active ingredient to a respiratory system of a subject in need thereof by inhalation. For example, the actual concentration of PFD formulated in the presence of the lipid carrier was detected in lung and blood, and the level was almost identical to formulated concentration of PFD as compared to other carriers.

In at least one embodiment, the medication described in the present disclosure comprises the active ingredient for treating an idiopathic pulmonary fibrosis (IPF).

In at least one embodiment, the medication comprising the active ingredient is mixed with the lipid carrier and atomized to an aerosol by atomizer. In at least one embodiment, the atomizer described in the present disclosure is an ultrasonic atomizer.

In at least one embodiment, the lipid carrier comprises Smoflipid®, Lipofundin®, Lipovenoes®, Lipovenos®, Intralipid®, Lipoplus®, or any combination thereof. In some embodiments, the lipid has a concentration of 10% to 20% (e.g., 10%, 11%, 12%, 13%, 14%, 15%, 16%, 17%, 18%, 19%, and 20%) based on a total volume of the lipid carrier. In at least one embodiment, the lipid carrier comprises soybean oil, medium-chain fatty acid, olive oil, fish oil, ω-3 oil, glycerol, lecithin, α-tocopherol, sodium oleate, palmitate, or any combination thereof. In some embodiments, the soybean oil has a concentration of 0% to 20% (e.g., 0%, 1%, 2%, 3%, 4%, 5%, 6%, 7%, 8%, 9%, 10%, 11%, 12%, 13%, 14%, 15%, 16%, 17%, 18%, 19%, and 20%). In some embodiments, the medium-chain fatty acid has a concentration of 0% to 20% (e.g., 0%, 1%, 2%, 3%, 4%, 5%, 6%, 7%, 8%, 9%, 10%, 11%, 12%, 13%, 14%, 15%, 16%, 17%, 18%, 19%, and 20%) based on a total volume of the lipid carrier. In some embodiments, the olive oil has a concentration of 0% to 10% (e.g., 0%, 1%, 2%, 3%, 4%, 5%, 6%, 7%, 8%, 9%, and 10%) based on a total volume of the lipid carrier. In some embodiments, the fish oil has a concentration of 0% to 5% (e.g., 0%, 1%, 2%, 3%, 4%, and 5%) based on a total volume of the lipid carrier. In some embodiments, the ω-3 oil has a concentration of 0% to 3% (e.g., 0%, 1%, 2%, and 3%) based on a total volume of the lipid carrier. In some embodiments, the glycerol has a concentration of 0% to 5% (e.g., 0%, 1%, 2%, 3%, 4%, and 5%) based on a total volume of the lipid carrier. In some embodiments, the lecithin has a concentration of 0% to 2% (e.g., 0%, 1%, and 2%) based on a total volume of the lipid carrier. In some embodiments, the α-tocopherol has a concentration of 0.01% to 0.15% (e.g., 0.01%, 0.02%, 0.03%, 0.04%, 0.05%, 0.06%, 0.07%, 0.08%, 0.09%, 0.10%, 0.11%, 0.12%, 0.13%, 0.14%, and 0.15%) based on a total volume of the lipid carrier. In some embodiments, the sodium oleate has a concentration of 0.01% to 0.5% (e.g., 0.01%, 0.02%, 0.03%, 0.04%, 0.05%, 0.06%, 0.07%, 0.08%, 0.09%, 0.10%, 0.11%, 0.12%, 0.13%, 0.14%, 0.15%, 0.16%, 0.17%, 0.18%, 0.19%, 0.20%, 0.21%, 0.22%, 0.23%, 0.24%, 0.25%, 0.26%, 0.27%, 0.28%, 0.29%, 0.31%, 0.32%, 0.33%, 0.34%, 0.35%, 0.36%, 0.37%, 0.38%, 0.39%, 0.41%, 0.42%, 0.43%, 0.44%, 0.45%, 0.46%, 0.47%, 0.48%, 0.49%, and 0.50%) based on a total volume of the lipid carrier.

In some embodiments, the buffer presents in the mixture is a solution having a pH value of about 6.0 to about 8.0 (e.g., 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, or 8.1), and the lipid carrier presents in the mixture in an amount ranging from volume percentage 0.5% to 40% (e.g., 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, and 40%).

Many examples have been used to illustrate the present disclosure. The examples below should not be taken as a limit to the scope of the present disclosure.

### EXAMPLES

For a more detailed description of the present disclosure, the method and the system for delivering an active ingredient by inhalation is provided and described in detail with reference to the following examples. The materials used in the present disclosure but unannotated herein are commercially available.

### Materials

(1) Lipoplus® 20% (LP):
   1000 ml of emulsion contains: 100.0 g medium-chain triglycerides, 80.0 g refined soybean oil, 20.0 g ω-3-acid triglycerides;
   content of triglycerides: 200 mg/mL (20%); and
   content of necessary fatty acid contains: 38.4 to 46.4 g/L linoleic acid (ω-6), 4.0 to 8.8 g/L α-linoleic acid (ω-3), 8.6 to 17.2 g/L eicosapentaenoic acid and docosahexaenoic acid (ω-3).
(2) Phosphate buffered saline (PBS); and
(3) Pirfenidone (PFD).

The chemicals used in the present disclosure were listed in table 1, and the instruments used in the present disclosure were indicated in table 2.

TABLE 1
list of chemicals used in the present disclosure.

| Chemical | Cas No. | Brand | Catalog No, |
|---|---|---|---|
| Lipoplus ® 20% | | B. BRAUN | 193428081 |
| Pirfenidone (PFD | | MedChemExpress | HY-B0673 |
| Anticoagulant Citrate | | | 451081 |
| Dextrose Solution | | | |
| Methanol | 67-56-1 | AENCORE | ME0606 |
| Perchloric acid | | | |
| Acetonitrile | 75-05-8 | AENCORE | AE0627 |
| Acetic acid | 64-19-7 | Sigma-aldrich | 33209 |

TABLE 2
list of instruments used in the present disclosure.

| Equipment | Brand | Model |
|---|---|---|
| Aerodynamic Particle Sizer (APS) | TSI | model 3321 |
| Scanning Mobility Particle Sizer | TSI | model 3938L88 |
| (SMPS) | | |
| Nebulizer | APEX | PY 001 |
| Vortex | TAITEC | μT-12 |
| Centrifuge | Eppendorf | Centrifuge 5904 R |
| Triple Quadrupole mass spectrometer | API | 2000 ™ System |
| C18 column | Agilent | Eclipse Plus C18 |

### Example 1: Atomizing Characteristic Evaluation

### Production Method

Phosphate buffered saline (PBS) was used and added in an atomizer, then atomized into an aerosol.

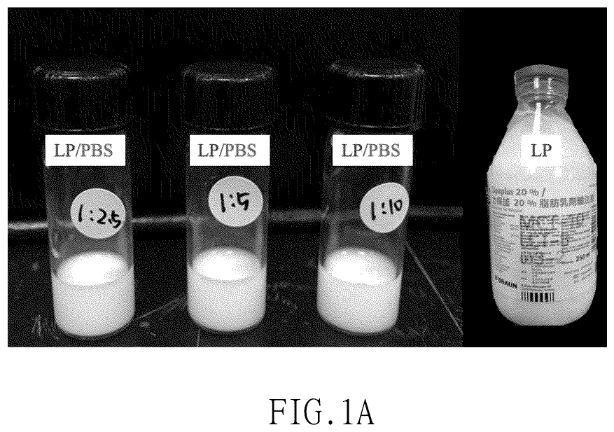

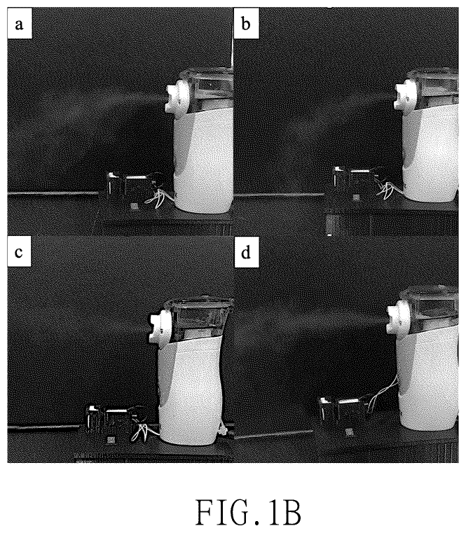

Lipoplus® was used and mixed with phosphate buffered saline (PBS). The mixture as shown in FIG. 1A was added in an atomizer and then atomized into an aerosol.

### Control Group (Ctrl. 1)—No-Lipoplus® Group;

$$Exp . 1 - 1 - {Lipolus}^{®} : PBS = 1 : 2.5 ( v / v ) ; \\ Exp . 1 - 2 - {Lipoplus}^{®} : PBS = 1 : 5 ( v / v ) ; \\ Exp . 1 - 3 - {Lipoplus}^{®} : PBS = 1 : 10 ( v / v ) .$$

The mixtures of Ctrl. 1 and Exp. 1-1 to 1-3 were put into an atomizer (APEX Mobi Mesh Portable Nebulizer, PY001) and then atomized into an aerosol respectively, and observe whether the atomizer can spray them smoothly. The atomizing characteristics thereof were evaluated.

The results are shown in FIG. 1B, it can be seen that Exp. 1-2 (panel c in FIG. 1B) can spray the aerosol smoothly, thus has good atomizing characteristic.

### Example 2. Assessment of Aerosol Particle Size for Drug Delivery Carriers

Large and small aerosol particle sizes were analyzed using two instruments. For example, aerodynamic particle sizer (APS; TSI model 3321) was employed to measure the size of large particles. For small particles, we used scanning mobility particle sizer (SMPS; TSI model 3938188) to measure the size. The two instruments used for measuring particle size were limited to detection of particle count. Therefore, our methodology involved briefly activating the instruments for one second followed by immediate deactivation. In between experimental group analysis, the nebulizer was cleaned and rinsed with alcohol and water followed by adding sample of new group, preventing any potential cross-contamination.

The experimental groups set for the example 2 were identical to the example 1 in the presence of PFD. As shown in table 3, the results measured by APS or SMPS indicate the aerosol particle size of PBS was found to be within the range of 857.6±1.3 nm. Specifically, when diluted at LP/PBS=1/10, the aerosol particle size was measured at 849.3±1.31 nm. For LP/PBS=1/5, the size ranged around 871.8±1.33 nm, and at LP/PBS=1/2.5, it was approximately 906.3±1.36 nm. These results illustrate that as the dilution ratio increases, aerosolized particles become smaller. For instance, particles at LP/PBS=1/2.5 were approximately 50 nm bigger than the other two dilution concentrations. Moreover, after adding PFD, a consistent reduction in particle size was observed in the measurements of larger particles. In contrast, only a marginal increase in particle size in the measurements of smaller particles was observed. The adding of PFD merely demonstrated minimal influence on particle size. This leads to an assumption that the predominant factor affecting particle size variation lies in the differences in dilution ratios.

TABLE 3
Aerosol particle sizes in the presence of PFD measured by APS (large
aerosol particle sizes) or SMPS (small aerosol particle sizes)

| | PFD | APS (Mean ± | SMPS (Mean ± |
|---|---|---|---|
| Dilution ratio (v/v) | (mg/mL) | SD (nm)) | SD (nm)) |
| PBS | — | 857.6 ± 1.30 | 87.5 ± 1.98 |
| LP/PBS = 1/10.0 | — | 849.3 ± 1.31 | 97.1 ± 2.18 |
| LP/PBS = 1/5.0 | — | 871.8 ± 1.33 | 97.4 ± 2.34 |
| LP/PBS = 1/2.5 | — | 906.3 ± 1.36 | 112.6 ± 2.32 |
| LP/PBS = 1/5.0 | 2 | 859.5 ± 1.32 | 106.9 ± 2.36 |
| LP/PBS = 1/5.0 | 3 | 853.1 ± 1.33 | 91.5 ± 2.36 |
| LP/PBS = 1/2.5 | 2 | 898.8 ± 1.35 | 114.1 ± 2.41 |

### Example 2: Spray Simulation Testing

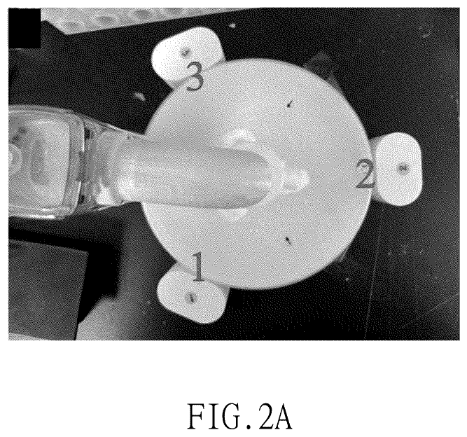

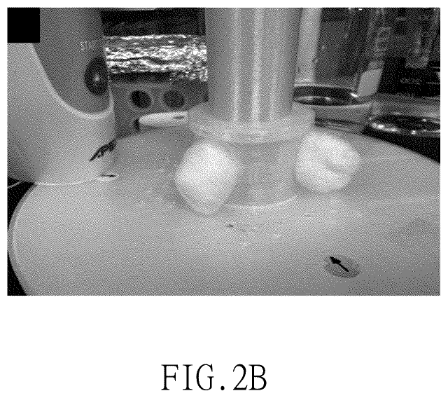

For each group, in the presence of PFD at 2 mg/mL as indicated in table 4, a quantity of dry cotton was initially weighed (approximately 0.251 g). Subsequently, the liquid was introduced into the nebulizer, and the cotton was placed within the spray orifice, as illustrated in FIG. 2A and FIG. 2B. Following the spray procedure, the cotton's damp weight was measured. The cotton was then immersed in 2 mL of methanol for one day, and the resulting solution was collected for further analysis.

TABLE 4
Condition of spray simulation testing

| | Dilution | PFD | Spraying |
|---|---|---|---|
| Solution | ratio (v/v) | (mg/mL) | volume (mL) |
| PBS | | 2 | 2 |
| LP/PBS | ⅕ | 2 | 2 |
| | 1/10 | 2 | 2 |

The primary objective of example 2 is to utilize cotton as a surrogate for mouse models to test whether the PFD, following atomizing, can reach the spray area, be absorbed by cotton, and ascertain the drug concentration for subsequent animal experiments. A comparison was made among three liquid formulations with identical drug amounts (PFD at 2 mg/mL): LP/PBS=1/5, LP/PBS=1/10, and PBS.

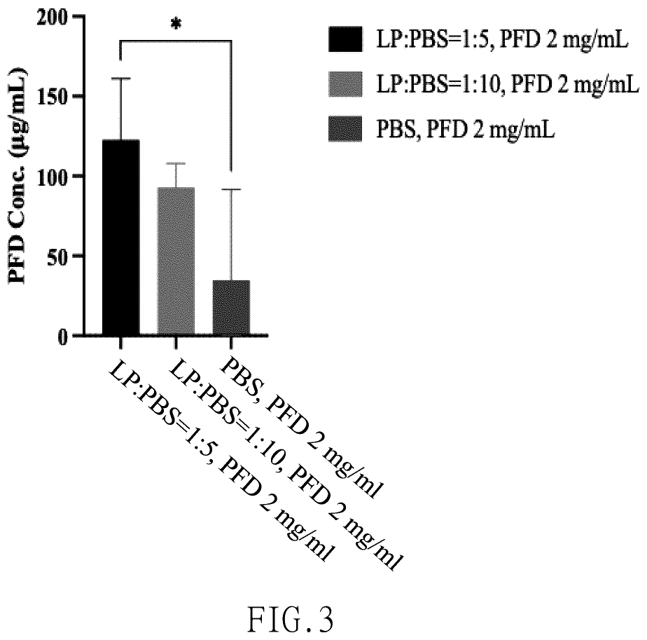

Under the same drug amount (PFD at 2 mg/mL), aerosol delivery to cotton produced an average PFD concentration of 101.082 μg/mL at LP:PBS=1:5, 61.980 μg/mL at LP:PBS=1:10, and significantly reduced to 23.120 μg/mL for the PBS group (as depicted in FIG. 3). The results (N=4) indicate that the quantity of PFD absorbed by cotton decreases with a reduction in LP. Therefore, solely using PBS as a solvent is not considered, and further animal experiments were used to compare the two sets of lipid emulsion dilution ratios.

### Example 3: Animal Experiment

### Production Method

PFD and Dimethyl sulfoxide (DMSO) were used. PFD was dissolved in 10 mg/ml of DMSO The mixture was used to make a calibration curve by High Performance Liquid Chromatography (HPLC).

PFD and Lipoplus® were used. PFD was dissolved in Lipoplus®-PBS (Exp. 1-2) to a concentration of 2 mg/mL. The mixture was added in an atomizer, then atomized to an aerosol. The aerosol was adsorbed by the cotton for extracting, and the extract was then used for HPLC analysis.

### Control Group (Ctrl. 2)—No-Atomization Group;

$$Exp . 2 - PFD in {Lipoplus}^{®} : PBS = 1 : 5 ( v / v ) .$$

The above experimental groups were used to determine whether the drugs can be dissolved in different formulations and atomized.

### Compound Preparation

The concentration was set at LP/PBS=1/5 (v/v) with a PFD at 2 mg/mL, a total spray volume of 2 mL. The experimental procedure entails the measurement of 4 mg of the compound, which is then incorporated into 0.34 mL of LP. Then, the drug and LP were mixed by a vortex. Subsequently, 1.66 mL of phosphate-buffered saline (PBS) is introduced into the blend, and this composite was also subjected to vortex mixing and for further utilization.

### Blood Processing

For the handling of blood samples, blood was blended with sodium citrate in a ratio of whole blood to sodium citrate as 9:1 (v/v). Subsequently, the mixture is set to stand for a duration of 30 minutes. Following this period, the sample was subjected to centrifugation at 1500×g for a duration of 15 minutes at a controlled temperature of 4° C. The resultant supernatant was then collected and reserved for subsequent utilization such as protein removal.

### Tissue Homogenization Procedure

After the removal of the lung tissue, it was weighed and subsequently introduced into methanol. For every 100 mg of tissue, 300 μL of methanol was added. Additionally, 5 mm diameter zirconium beads were incorporated into the mixture. Employing a vibrational homogenizer, the procedure consisted of running the homogenizer at a speed of 2500 rpm, with 30 seconds of shaking followed by a 10-second rest. This cycle was repeated six times (3 minutes for each time). The resultant supernatant was then collected and reserved for subsequent utilization such as protein removal.

### Protein Removal

For the protein removal, 300 μL of plasma/tissue fluid was acquired and supplemented with 100 μL of 10% perchloric acid. This mixture was subjected to a vibrational homogenizer at 1800 rpm for a duration of 3 minutes (without zirconium beads). The sample was then centrifuged at 12,000×g for 10 minutes at a temperature of 4° C. The resultant supernatant was then collected and stored at −80° C. followed by high-performance liquid chromatography (HPLC) analysis.

### Method for Drug Concentration Analysis

Utilizing high-performance liquid chromatography (HPLC), a calibration curve for quantification was established within the concentration range of 0.125-2 μg/mL. The mobile phase consisted of a mixture of acetonitrile and water in a ratio of 23:77 (v/v) with the addition of 0.2% acetic acid. A C18 column was employed. Prior to analysis, the mobile phase was degassed using ultrasonic agitation for 30 minutes. A sample volume of 180 μL was introduced into the autosampler, and the analysis was conducted under the following conditions:

Calibration Curve Preparation Method: The PFD in DMSO at a concentration of 10 mg/mL was diluted to 1 mg/mL with pure water filtered with a 0.22 μm filter. Further dilutions should be made to reach concentrations of 2 μg/mL, 0.5 g/mL, and 0.125 μg/mL. These prepared solutions are then transferred into sample vials and are ready for analysis. For the sample analysis, the following parameters were applied: the sample temperature was maintained at 25° C., the column temperature was set to 45° C., the flow rate was adjusted to 1 mL/min, the detection wavelength was fixed at 310 nm, and the injection volume was 100 μL. The total analysis time was around 15 minutes, and PFD was detected within the time frame of 8 to 9 minutes.

### Experimental Method

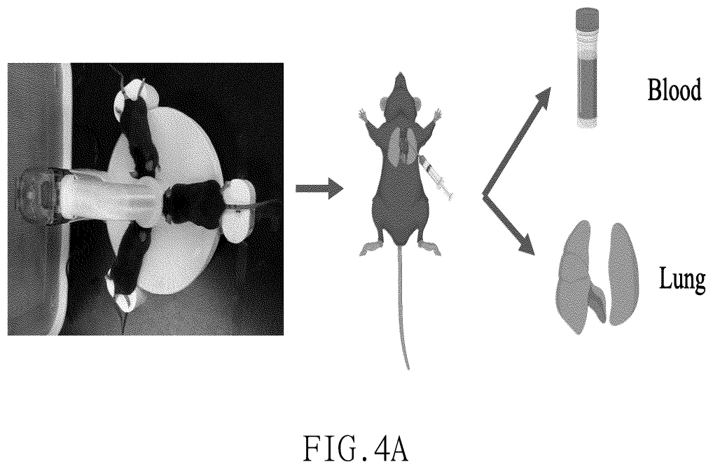

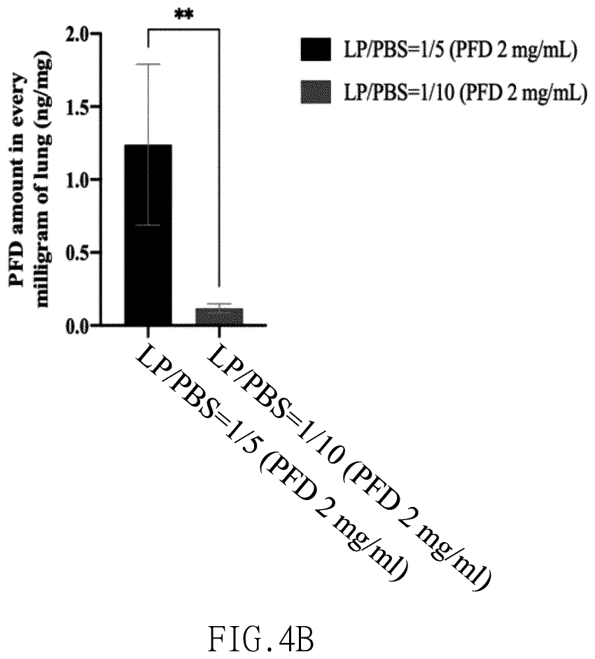

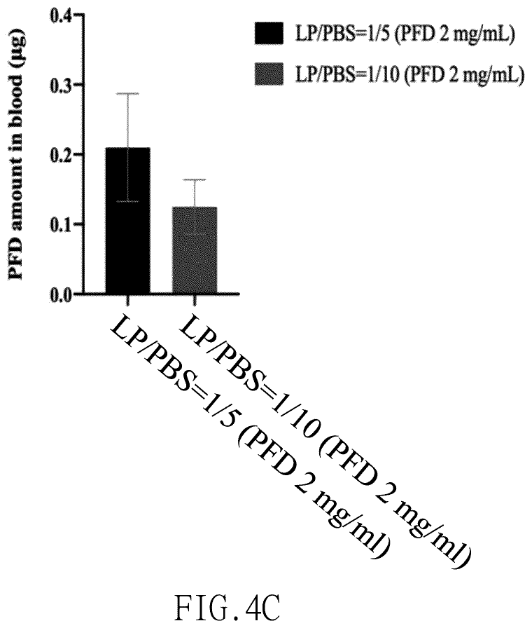

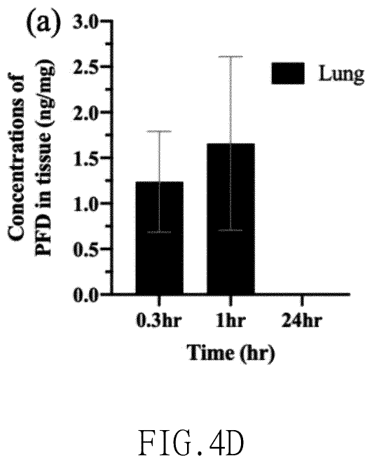

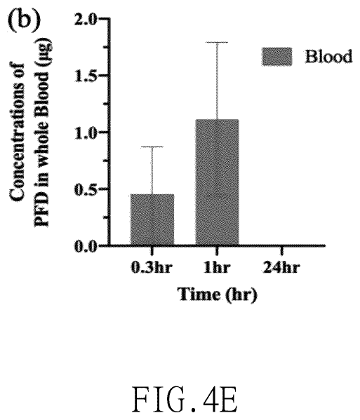

In Example 3, animal experimentation were firstly simulated with cotton as indicated in FIG. 2A to 2B, and the cottons were placed in the position where the mice were placed as shown in FIG. 4A for the animal experiments.

First, weigh a dry cotton; add the mixture of Exp. 2 into an atomizer (APEX Mobi Mesh Portable Nebulizer, PY001) to atomized into an aerosol, then spray the aerosol on the dry cottons at different positions 1 to 3 respectively, and weigh the wet cotton; put the wet cotton in 2 mL methanol to obtain a PFD-containing solution; and collect the PFD-containing solution for HPLC analysis.

FIG. 2B and FIG. 4A show the position of the cotton for HPLC analysis. The equations used for calculating Pirfenidone (PFD) concentration for spray simulation or in different tissues were obtained from calibration curve as mentioned above. Table 5 shows higher spray amount at position 1 and uniform spray amount at positions 2 and 3. It can be seen that the aerosol of the present disclosure can successfully absorbed by the cotton, and the signal of PFD can be observed in HPLC analysis of the collected liquid from the cotton. That is, PFD can be dissolved using Lipoplus® as a solvent then be atomized to an aerosol using an atomizer, the aerosol can be absorbed by cotton that in place of mice, and the PFD in cotton could be detected at concentrations range from 95.6714 to 53.7478 g.

For the animal experiments, immediately after spraying the drug (approximately 20 mins), the mice were sacrificed to compare the effects of the two dilution rations on the delivery of the drug into the animals. As shown in FIG. 4B, the group treated with LP/PBS=1/5 indicated the average amount of PFD measured was 1.239 ng/mg vs. 0.115 ng/mg, being treated with LP/PBS=1/10, in the lung. On the other hand, as shown in FIG. 4C, results measured in the blood show the average amount was 0.209 μg vs. 0.125 μg (LP/PBS=1/5 vs. LP/PBS=1/10). As the LP dilution decreases, the PFD concentration measured in the lungs differs by almost ten times although the total amount of PFD added was the same. Therefore, the dilution ratio of LP/PBS=1/5 (PFD 2 mg/mL) was used for subsequent drug retention in mice.

Moreover, mice were treated with LP/PBS=1/5 (PFD 2 mg/mL). After aerosol administration, mice were sacrificed at three time points, 0.3 hr, 1 hr, and 24 hr, to collect tissues and blood for testing. FIG. 4D and FIG. 4E show that actual concentration of PFD in lung and blood are almost identical to formulated concentration of PFD. It was shown that in lung, the average PFD concentration measured at 0.3 hr was 1.239 ng/mg, which further increased to 1.65 ng/mg after 1 hour. In the blood, an average PFD measured at 0.3 hr was 0.45 μg/mL, which increased to 1.114 μg/mL after 1 hour. PFD concentration in lung and blood were not detectable one day later, showing that PFD has been metabolized and eliminated from the body.

TABLE 5
Characteristics of cotton at different positions

| | | | Dry | HPLC con- | |
|---|---|---|---|---|---|
| | | | weight − | centration | |
| Position | Dry | Wet | Wet | of PFD | ×dilution |
| of cotton | weight (g) | weight (g) | weight(g) | (μg/mL) | multiple |
| 1 | 0.2214 | 0.4222 | 0.2008 | 95.6714 | 191.3428 |
| 2 | 0.2386 | 0.3749 | 0.1363 | 53.3952 | 106.7905 |
| 3 | 0.2551 | 0.3924 | 0.1372 | 53.74785 | 107.4957 |

The results indicate that PFD as the active ingredient can be dissolved in lipid carriers, and atomized; and the method and lipid carrier for delivering an active ingredient by inhalation effectively increases the concentration of active ingredient in the respiratory system.

Altogether, these above results indicate that the method, system, and lipid carrier for delivering an active ingredient by nasal inhalation effectively increases the concentration of active ingredient in the lower lobe of a lung.

Those skilled in the art will readily observe that numerous modifications and alterations of the method and system may be made while retaining the teachings of the invention. Accordingly, the above disclosure should be construed as limited only by the metes and bounds of the appended claims.

What is claimed is:

1. A method for delivering an active ingredient by inhalation, comprising:
atomizing a mixture of the active ingredient, an aqueous solution, and a lipid carrier into an aerosol; and
administering an effective amount of the active ingredient to a subject in need thereof by inhalation of the aerosol into a respiratory system of the subject.

2. The method of claim 1, wherein the aqueous solution comprises a buffer, and the lipid carrier comprises a lipid emulsion.

3. The method of claim 2, wherein the lipid emulsion comprises at least one lipid selected from the group consisting of Smoflipid®, Lipofundin®, Lipovenoes®, Lipovenos®, Intralipidand®, Lipoplus®, and any combination thereof.

4. The method of claim 3, wherein the lipid has a concentration of about 10% to about 20% based on a total volume of the lipid carrier.

5. The method of claim 3, wherein the lipid emulsion comprises the Lipoplus®.

6. The method of claim 2, wherein the lipid carrier comprises at least one ingredient selected from the group consisting of soybean oil, medium-chain fatty acid, olive oil, fish oil, ω-3 oil, glycerol, lecithin, α-tocopherol, sodium oleate, palmitate, and any combination thereof.

7. The method of claim 6, the soybean oil has a concentration less than 20% based on the total volume of the lipid carrier, the medium-chain fatty acid has a concentration less than 20% based on the total volume of the lipid carrier, the olive oil has a concentration less than 10% based on the total volume of the lipid carrier, the fish oil has a concentration less than 5% based on the total volume of the lipid carrier, the ω-3 oil has a concentration of less than 3% based on the total volume of the lipid carrier, the glycerol has a concentration less than 5% based on the total volume of the lipid carrier, the lecithin has a concentration of less than 2% based on the total volume of the lipid carrier, the α-tocopherol has a concentration of 0.01% to 0.15% based on the total volume of the lipid carrier, the sodium oleate has a concentration of 0.01% to 0.5% based on the total volume of the lipid carrier, and the palmitate has a concentration of 0.01% to 0.5% based on the total volume of the lipid carrier.

8. The method of claim 1, wherein the respiratory system comprises a respiratory tract or a lung.

9. The method of claim 1, wherein the respiratory system comprises oral cavity, nasal cavity, pharynx, larynx, trachea, carina, primary bronchi, secondary bronchus, tertiary bronchi, bronchioles, alveoli, or any combination thereof.

10. The method of claim 2, wherein the buffer is a solution having a pH value of about 6.0 to about 8.0.

11. The method of claim 10, wherein the buffer is a saline solution comprising at least one pharmaceutically acceptable salt of borate, citrate, bicarbonate, carbonate, glutamate, lactate, malate, phosphate, acetate, alginate, aspartate, sulfonate, fumarate, lactobionate, laurate, maleate, or palmitate.

12. The method of claim 10, wherein the buffer is an organic compound solution comprises at least one pharmaceutically acceptable amine of glycine, methionine, monoethanolamine, diethanolamine, 2-Amino-2-hydroxymethyl-propane-1,3-diol, triethylamine, procaine, dibenzylamine, N-benzyl-β-phenethylamine, 1-ephenamine, N,N′-dibenzylethylene-diamine, dehydroabietylamine, N-ethylpiperidine, benzylamine, or dicyclohexylamine.

13. The method of claim 1, wherein the active ingredient is a predetermined class of a small molecule with molecular weight≤1000 daltons.

14. The method of claim 13, wherein the small molecule comprises a natural ingredient, an artificial ingredient, or an autologous ingredient.

15. The method of claim 1, wherein the active ingredient is at least one selected from the group consisting of Pirfenidone (PFD), Nintedanib, mRNA, steroid, anticoagulants, immunosuppressive drugs/agents, endothelin receptor antagonist, phosphodiesterase inhibitor, colchicine, chemotherapeutic agents, peptide, and protein.

16. The method of claim 15, wherein the active ingredient is a large molecule comprising a natural ingredient, an artificial ingredient, or an autologous ingredient.

17. The method of claim 1, wherein the lipid carrier comprises a lipid emulsion, the buffer is a solution having a pH value of about 6.0 to about 8.0, and the lipid carrier presents in the mixture in an amount ranging from volume percentage about 0.5% to about 40%.

18. The method of claim 1, wherein the aerosol is atomized by a pharmaceutically acceptable atomizer, nebulizer, or inhaler.

19. A delivering system for delivering an active ingredient to a respiratory system of a subject in need thereof by inhalation, comprising
a mixture of the active ingredient, an aqueous solution, and a lipid carrier; and
a pharmaceutically acceptable atomizer, nebulizer, or inhaler for atomizing the mixture into an aerosol.

20. The delivering system of claim 19, wherein the aqueous solution comprises a buffer, and the lipid carrier comprises a lipid emulsion.
